SEQUENCE LISTING (2 sequences)

SEQ ID NO: 1 - protein, 478 aa, Homo sapiens
NPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 2 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

(19) United States
(12) Patent Application Publication
(10) Pub. No.: US 2014/0315826 A1
(43) Pub. Date: Oct. 23, 2014

(54) POLYMERIC CONJUGATES OF C-1 INHIBITORS

(71) Applicant: Belrose Pharma, Inc., Princeton, NJ (US)

(72) Inventors: Hong Zhao, Edison, NJ (US); Karen Yang, Edison, NJ (US)

(73) Assignee: Belrose Pharma, Inc., Princeton, NJ (US)

(21) Appl. No.: 14/358,988

(22) PCT Filed: Mar. 15, 2013
(86) PCT No.: PCT/US2013/032135

§ 371 Date: May 16, 2014

Related U.S. Application Data
(60) Provisional application No. 61/612,213, filed on Mar. 16, 2012.
(60) Provisional application No. 61/749,840, filed on Jan. 7, 2013.
(60) Provisional application No. 61/749,842, filed on Jan. 7, 2013.
(60) Provisional application No. 61/749,848, filed on Jan. 7, 2013.

(51) Int. Cl. A61K 38/57; A61K 47/48

(52) U.S. Cl. CPC A61K 38/57; A61K 47/48215
USPC 514/20.1; 530/395

(57) ABSTRACT

Polymer conjugates containing a C1-inhibitor having at least one substantially non-antigenic polymer covalently attached to the C1-inhibitor via glycan group of the C1 inhibitor is provided. In addition, methods of making the conjugates as well as methods of treatment using the conjugate of the present invention are also provided.

### CROSS-REFERENCE TO RELATED APPLICATIONS

This application claims the benefit of priority from U.S. Provisional patent application Ser. Nos. 61/612,213 filed Mar. 16, 2012, and 61/749,840, 61/749,842 and 61/749,848 filed Jan. 7, 2013, the contents of each of which are incorporated herein by reference.

### FIELD OF INVENTION

The present invention relates to polymer conjugates containing a C1-inhibitor having at least one substantially non-antigenic polymer covalently attached to the oxidized OH in glycan of C1-inhibitor via a hydrazine, hydrazine bond, or amine via reductive amination, and uses thereof

### BACKGROUND OF THE INVENTION

C1-inhibitor is a normal constituent of human plasma and belongs to the group of serine protease inhibitors (serpins). One type of C1-inhibitor, C1 esterase inhibitor, is a soluble, single-chain glycoprotein containing 478 amino acid residues. The plasma concentration of C1-esterase inhibitor in a healthy human body is approximately 270 mg/L. C1-inhibitor is a down-regulator of inflammatory processes in blood. Unlike most family members, C1-inhibitor has a 2-domain structure: the C-terminal serpin domain, which is similar to other serpins, and the N-terminal domain. Structural analysis showed the N-terminal is highly glycosylated leaving the C-terminal more susceptible to reactive binding sites.

Deficiency of this protein is associated with hereditary angioedema or angioneurotic edema, or swelling due to leakage of fluid from blood vessels into connective tissue. Symptoms include swelling of the face, mouth and/or airway that occurs spontaneously or by minimal triggers (such as mild trauma). Such swelling can also occur in any part of the body. In some cases, the levels of C1-inhibitor are low, while in others the protein circulates in normal amounts but it is dysfunctional. In addition to the episodes of facial swelling and/or abdominal pain, it also can cause more serious or life threatening indications, such as autoimmune diseases or lupus erythematosus.

In people with hereditary angioedema, Cinryze® is used to prevent attacks of angioedema, when the C1-esterase inhibitor does not function properly or occurs in low levels, while Berinert® is used to treat attacks of angioedema. Cinryze® is administered at a dose of 1,000 units intravenously at 1 mL/min for 10 min, every 3 or 4 days for routine prophylaxis against angioedema attacks, and Berinert® is administered at a dose of 20 units per kg body weight intravenously at 4 mL/min. Accordingly, non-compliance is a major obstacle to the effective delivery of the C1-esterase inhibitor.

In spite of previous efforts, there is still an unmet need for an improved form of a C1-inhibitor. For example, it would be beneficial to provide long acting C1-inhibitors so that the frequency of dosing could be reduced. The present invention addresses this need.

### SUMMARY OF THE INVENTION

The present invention provides polymer conjugates containing a C1-inhibitor having at least one substantially non-antigenic polymer covalently attached to the oxidized OH in glycan of C1-inhibitor via a hydrazine, hydrazine bond, or amine via reductive amination. In another aspect of the invention, more than one polymer is attached to glycan of C1 inhibitor via independently different types of chemical bonds including permanent or releasable bonds.

Methods of making the conjugates as well as methods of treatment using the conjugates of the present invention are also provided. Advantages will be apparent from the following description.

For purposes of the present invention, the term “residue” shall be understood to mean that portion of a conjugate, to which it refers, e.g., amino acid, etc. that remains after it has undergone a substitution reaction with another conjugate.

For purposes of the present invention, the term “polymeric containing residue” or “PEG residue” shall each be understood to mean that portion of the polymer or PEG which remains after it has undergone a reaction with C1-inhibitor.

For purposes of the present invention, the term “alkyl” shall be understood to include straight, branched, substituted, e.g. halo-, alkoxy-, nitro-, C₁₋₁₂, but preferably C₁₋₄ alkyls, C₃₋₈ cycloalkyls or substituted cycloalkyls, etc.

For purposes of the present invention, the term “substituted” shall be understood to include adding or replacing one or more atoms contained within a functional group or conjugate with one or more different atoms.

For purposes of the present invention, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as napthyl; substituted aryls include moieties such as 3-bromo phenyl; aralkyls include moieties such as tolyl; heteroalkyls include moieties such as ethylthiophene; substituted heteroalkyls include moieties such as 3-methoxy-thiophene; alkoxy includes moieties such as methoxy; and phenoxy includes moieties such as 3-nitrophenoxy. Halo shall be understood to include fluoro, chloro, iodo and bromo.

The terms “effective amounts” and “sufficient amounts” for purposes of the present invention shall mean an amount which achieves a desired effect or therapeutic effect as such effect is understood by those of ordinary skill in the art.

### DETAILED DESCRIPTION OF THE INVENTION

In one aspect of the present invention, polymer conjugates of a C1-inhibitor having at least one substantially non-antigenic polymer covalently attached thereto via one of more glycan groups are provided.

In one embodiment, polymer conjugates are provided in which the substantially non-antigenic polymer is a polyalkylene oxide such as a polyethylene glycol.

In yet another embodiment, polymer conjugates are provided wherein the C1-inhibitor is a human C1 esterase inhibitor (C1-INH) or a polypeptide represented by SEQ ID NO: 1 or SEQ ID NO: 2.

In a further embodiment, polymer conjugates are provided in which one of the substantially non-antigenic polymers are attached to an aldehyde in glycan of C1 inhibitor, which is generated by oxidation of OH in the glycan.

In another aspect of the invention, polymer conjugates are provided in which one of the substantially non-antigenic polymers are attached via hydrazone or hydrazide bond through the oxidized glycan of C1 inhibitor.

In the present invention, one of more OH groups in glycan of C1 inhibitor is oxidized by employing an oxidizing agent to convert primary OH to an aldehyde. An activated polymer having a nucleophilic functional group able to conjugate with the oxidized C1 inhibitor allows site selective polymer conjugation.

The aldehyde moieties provide chemical selectivity over other chemical functional moieties to conjugate the polymer site specifically. Polymer conjugate via glycan provides an additional advantage over conjugation via one of the amino acids in the protein. Most binding or reacting domains which are responsible for the biological activities of the protein are located within the amino acid sequence, sometimes near the N-terminal or near the C-terminal of the protein. Glycans are located on the outside of the main amino acid sequences and thus, without being bound by any theory, polymer conjugation on the glycan affects the binding or biological activities of the protein least. The site selective conjugation provides a more consistent and uniform product often with higher biological activity as compared to other conjugation techniques.

The polymer conjugates of the invention retain at least about 20% of the biological activity of the native C1-inhibitor and preferably about 40-80% of the biological activity of the native C1-inhibitor.

Polymer conjugates are provided having Formula (I) or (I′):

[PEG-(L)ₘ-(C(═Y)—NH)ₙ]ₚ—(X)_{p′}—C1-inhibitor (I) or

[PEG-(L)ₘ-(C(═Y)—NH)ₙ]ₚ—(X)_{p′}—C1-inhibitor-(X′)_{q′}-[(C(═Y′)—NH)_{n′}-(L′)_{m′}-PEG]_{q′} (I′)

wherein

C1 inhibitor is bonded to PEG via an amine from PEG through glycan site;

PEG is a linear, branched or multi-arm poly(ethylene glycol) having a terminal group —(CH₂CH₂O)—;

Y or Y′ is independently O or S;

L or L′ is independently a linker or functional group suitable to react with thiol; (m) or (m′) is independently 0 or 1;

(n) or (n′) is independently zero or a positive integer, preferably selected from 1, 2, 3, 4, 5, 6, 7, 8, 9, or 10;

(p) or (q) is independently a positive integer, preferably selected from 1, 2, 3, 4, 5, 6 or 7; and

X or X′ is NH, an amine from PEG hydrazine or PEG amine;

(p′) or (q′) is independently a positive integer same as (p) or (q), respectively, provided that (m), (m′), (n) and (n′) are not zero simultaneously.

In one aspect of the invention, in the polymer conjugate of Formula (I) or (I′) described above, (n) or (n′) is a positive integer selected from among 1, 2, 3, 4, 5, 6 or 7 and (p) or (q) is a positive integer selected from among of 1, 2 or 3.

In another embodiment, in the polymer conjugates of Formula (I) or (I′) described above, L or L′ is a bifunctional moiety which contains at least one chemically blocked or protected functional group and at least one reactive or an activated functional group which reacts with the first conjugation, polymer or C1 inhibitor.

### Polymers

In one preferred embodiment, the polymer conjugate described herein can employ a variety of water soluble polymers which have the following formula:

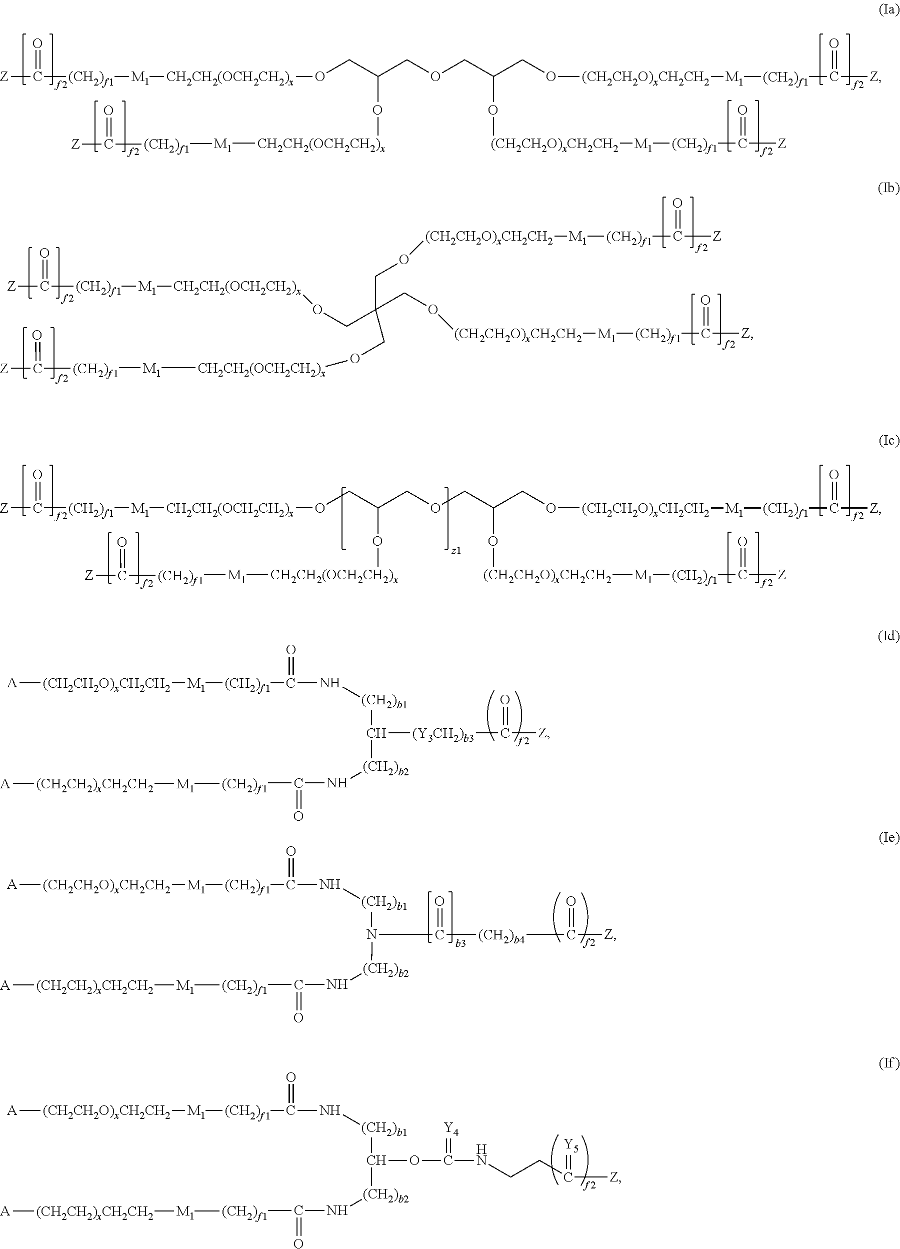

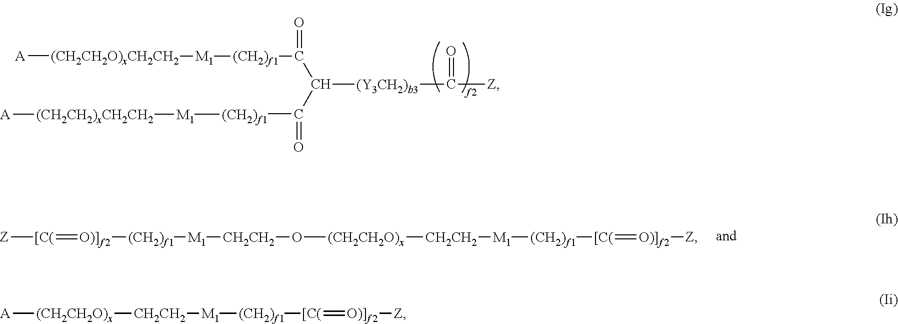

wherein

A is hydroxyl, NH₂, CO₂H, or C₁₋₆ alkoxy;

M₁ is O, S, or NH;

Y₃ is O, NR₅₁, S, SO or SO₂;

Y₄ and Y₅ are independently O, S or NR₅₁;

R₅₁, in each occurrence, is independently hydrogen, C₁₋₈ alkyl, C₁₋₈ branched alkyl, C₁₋₈ substituted alkyl, aryl, or aralkyl;

Z, in each occurrence, is independently OH, a leaving group, a targeting group, C₁₋₈ alkyl, C₁₋₈ alkoxy, or C1 inhibitor containing moiety;

(b1) and (b2) are independently zero or positive integers;

(b3) is zero or 1;

(b4) is a positive integer;

(f1) is zero or a positive integer of from about 1 to about 10;

(f2) is zero or 1;

(z1) is zero or a positive integer of from 1 to about 27;

(n) is a positive integer of from about 10 to about 2,300 so that the polymeric portion of the conjugate has the total number average molecular weight of from about 2,000 to about 100,000 daltons; and

all other variables are the same as previously defined; provided that one or more Z is a C1-inhibitor containing moiety.

In a certain embodiments, the molecular weight of the substantially non-antigenic polymer ranges from about 2,000 to about 60,000 daltons, preferably the molecular weight of the substantially non-antigenic polymer ranges from about 5,000 to about 50,000 daltons, and more preferably from about 20,000 to about 40,000 daltons.

In another embodiment, the substantially non-antigenic polymer is conjugated via a hydrazide, a hydrazone, or an amine bond.

According to the present invention, polymers contemplated within the conjugates described herein are preferably water soluble and substantially non-antigenic, and include, for example, polyalkylene oxides (PAO's). The conjugates described herein further include linear, branched, or multi-armed polyalkylene oxides. In one preferred aspect of the invention, the polyalkylene oxide includes polyethylene glycols and polypropylene glycols. More preferably, the polyalkylene oxide includes polyethylene glycol (PEG).

PEG is generally represented by the structure:

—(CH₂CH₂O)ₓ—

where (x) is a positive integer of from about 10 to about 2300 so that the polymeric portion of the conjugates described herein has a number average molecular weight of from about 2,000 to about 100,000 daltons.

The polyalkylene oxide has a total number average molecular weight of from about 2,000 to about 100,000 daltons, preferably from about 5,000 to about 60,000 daltons. The molecular weight of the polyalkylene oxide can be more preferably from about 5,000 to about 25,000 or from about 20,000 to about 45,000 daltons. In some particularly preferred embodiments, the conjugates described herein include the polyalkylene oxide having a total number average molecular weight of from about 30,000 to about 45,000 daltons. In one particular embodiment, a polymeric portion has a total number average molecular weight of about 40,000 daltons.

Alternatively, the polyethylene glycol can be further functionalized as represented by the structure:

-[C(═O)]_{f2}—(CH₂)_{f1}-M₁-CH₂CH₂(OCH₂CH₂)—O-A

wherein

M₁ is O, S, or NH;

(f1) is zero or a positive integer of from about 1 to about 10, preferably, 0, 1, 2, or 3, more preferably, zero or 1;

(f2) is zero or one;

(n) is a positive integer of from about 10 to about 2,300; and

A is hydroxyl, NH₂, CO₂H, or C₁₋₆ alkoxy.

In one embodiment, A is methoxy.

In certain embodiments, all four of the PEG arms can be converted to suitable activating groups, for facilitating attachment to other molecules (e.g., bifunctional linkers). Such conjugates prior to conversion include:

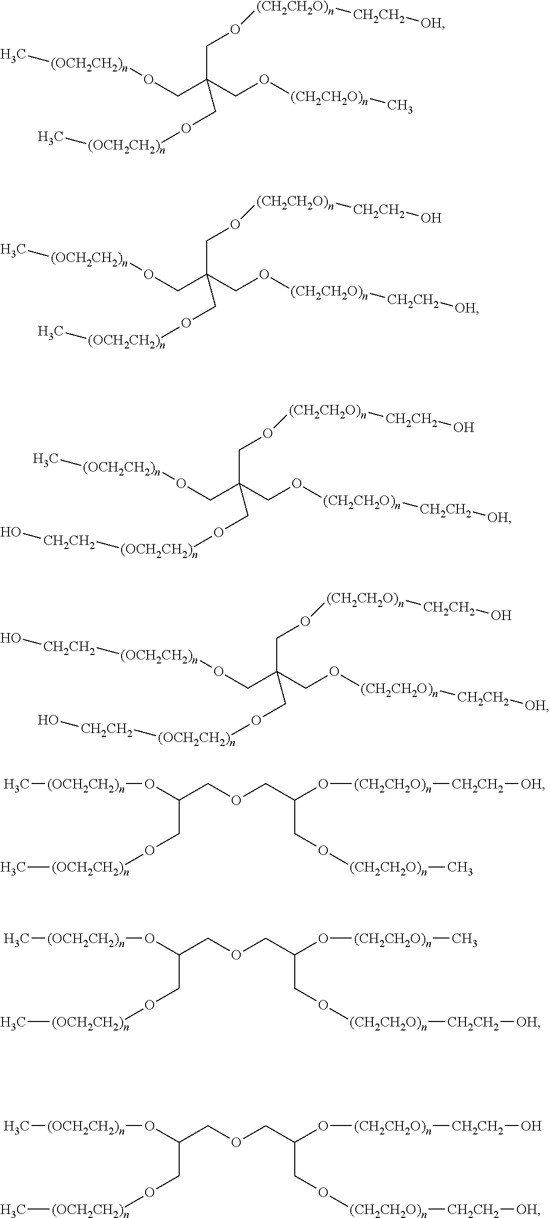

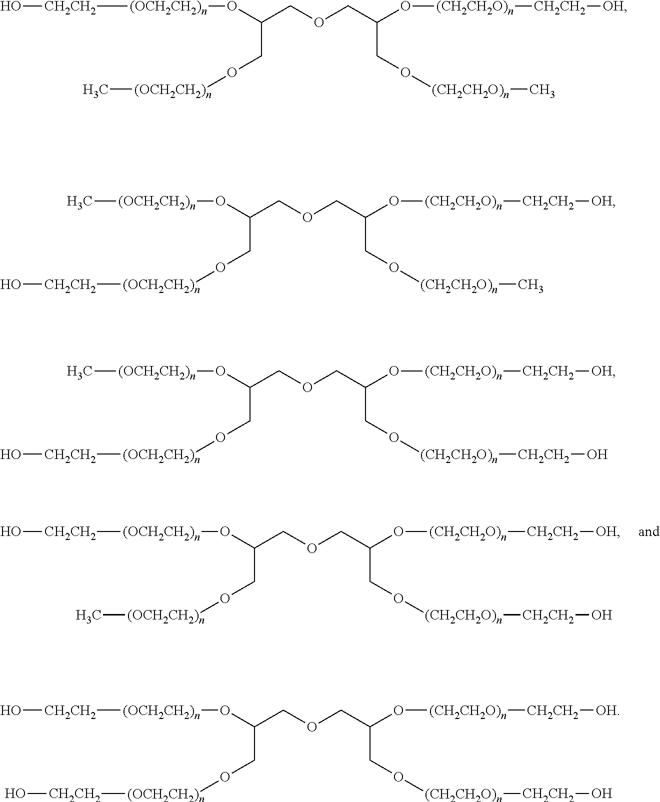

PEG should include an amine, a hydrazide or other aldehyde PEGylating linker.

In yet a further aspect of the invention, the polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In yet a further aspect and as an alternative to PAO-based polymers such as PEG, one or more effectively non-antigenic materials such as dextran, polyvinyl alcohols, carbohydrate-based polymers, hydroxypropylmethacrylamide (HPMA), polyalkylene oxides, and/or copolymers thereof can be used. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will appreciate that the foregoing list is merely illustrative and that all polymeric materials having the qualities described herein are contemplated. For purposes of the present invention, “substantially or effectively non-antigenic” means polymeric materials understood in the art as being nontoxic and not eliciting an appreciable immunogenic response in mammals.

### Linkers

In one aspect, the substantially non-antigenic polymer of the present invention is conjugated to C1-inhibitor via a hydrazide, a hydrazone, or an amine.

In one aspect, the substantially non-antigenic polymer of the present invention is conjugated to C1-inhibitor via a linking moieties or a bifunctional spacer.

In some embodiment, the bifunctional moieties contain a residue of a bifunctional spacer such as,
—[C(═O)]ᵥ(CR₂₂R₂₃)ₜ[C(═O)]_{v′}—,
—[C(═O)]ᵥ(CR₂₂R₂₃)ₜ—O[C(═O)]_{v′}—,
—[C(═O)]ᵥ(CR₂₂R₂₃)ₜ—NR₂₆[C(═O)]_{v′}—,
—[C(═O)]ᵥO(CR₂₂R₂₃)ₜ[C(═O)]_{v′}—,
—[C(═O)]ᵥO(CR₂₂R₂₃)ₜO[C(═O)]_{v′}—,
—[C(═O)]ᵥO(CR₂₂R₂₃)ₜNR₂₆[C(═O)]_{v′}—,
—[C(═O)]ᵥNR₂₁(CR₂₂R₂₃)ₜ[C(═O)]_{v′}—,
—[C(═O)]ᵥNR₂₁ (CR₂₂R₂₃)ₜO[C(═O)]_{v′}—,
—[C(═O)]ᵥNR₂₁ (CR₂₂R₂₃)ₜNR₂₆[C(═O)]_{v′}—,
—[C(═O)]ᵥ(CR₂₂R₂₃)ₜO—(CR₂₈R₂₉)_{t′}[C(═O)]_{v′}—,
—[C(═O)]ᵥCR₂₂R₂₃)ₜNR₂₆—(CR₂₈R₂₉)_{t′}[C(═O)]_{v′}—,
—[C(═O)]ᵥ(CR₂₂R₂₃)ₜS—(CR₂₈R₂₉)_{t′}[C(═O)]_{v′}—,
—[C(═O)]ᵥ(CR₂₂R₂₃)ₜO—(CR₂₈R₂₉)_{t′}[C(═O)]_{v′}—,
—[C(═O)]ᵥO(CR₂₂R₂₃)ₜNR₂₆—(CR₂₈R₂₉)_{t′}[C(═O)]_{v′}—,
—[C(═O)]ᵥO(CR₂₂R₂₃)ₜS—(CR₂₈R₂₉)_{t′}[C(═O)]_{v′}—,
—[C(═O)]ᵥNR₂₁ (CR₂₂R₂₃)ₜO—(CR₂₈R₂₉)_{t′}[C(═O)]_{v′}—,
—[C(═O)]ᵥNR₂₁(CR₂₂R₂₃)ₜNR₂₆—(CR₂₈R₂₉)_{t′}[C(═O)]_{v′}—,
—[C(═O)]ᵥNR₂₁ (CR₂₂R₂₃)ₜS—(CR₂₈R₂₉)_{t′}[C(═O)]_{v′}—,
—[C(═O)]ᵥ(CR₂₂R₂₃)CR₂₈R₂₉O)ₜNR₂₆)[C(═O)]_{v′}—,
—[C(═O)]ᵥ(CR₂₂R₂₃CR₂₈R₂₉O)ₜ[C(═O)]_{v′}—,
—[C(═O)]ᵥO(CR₂₂R₂₃CR₂₈R₂₉O)ₜNR₂₆[C(═O)]_{v′}—,
—[C(═O)]ᵥO(CR₂₂R₂₃CR₂₈R₂₉O)ₜ[C(═O)]_{v′}—,
—[C(═O)]ᵥNR₂₁ (CR₂₂R₂₃CR₂₈R₂₉O)ₜ NR₂₆[C(═O)]_{v′}—,
—[C(═O)]ᵥNR₂₁ (CR₂₂R₂₃CR₂₈R₂₉O)ₜ[C(═O)]_{v′}—,
—[C(═O)](CR₂₂R₂₃CR₂₈R₂₉O)ₜ(CR₂₄R₂₅)_{t′}[C(═O)]_{v′}—,
—[C(═O)]ᵥO(CR₂₂R₂₃CR₂₈R₂₉O)ₜ(CR₂₄R₂₅)_{t′}[C(═O)]_{v′}—,
—[C(═O)]ᵥNR₂₁ (CR₂₂R₂₃CR₂₈R₂₉O)ₜ(CR₂₄R₂₅)_{t′}[C(═O)]_{v′}—,
—[C(═O)]ᵥ(CR₂₂R₂₃CR₂₈R₂₉O)ₜ(CR₂₄R₂₅)_{t′}[C(═O)]_{v′}—,
—[C(═O)]ᵥ(CR₂₂R₂₃)ₜ(CR₂₄R₂₅CR₂₈R₂₉O)_{t′}[C(═O)]_{v′}—,
—[C(═O)]ᵥ(CR₂₂R₂₃)ₜ(CR₂₄R₂₅CR₂₈R₂₉O)_{t′}NR₂₆[C(═O)]_{v′}—,
—[C(═O)]ᵥO(CR₂₂R₂₃CR₂₈R₂₉O)ₜ(CR₂₄R₂₅)_{t′}O[C(═O)]_{v′}—,
—[C(═O)]ᵥO(CR₂₂R₂₃)ₜ(CR₂₄R₂₅CR₂₈R₂₉O)_{t′}[C(═O)]_{v′}—,
—[C(═O)]ᵥO(CR₂₂R₂₃)ₜ(CR₂₄CR₂₅CR₂₈R₂₉O)_{t′}NR₂₆[C(═O)]_{v′}—,
—[C(═O)]ᵥNR₂₁ (CR₂₂R₂₃CR₂₈R₂₉O)ₜ(CR₂₄R₂₅)_{t′}[C(═O)]_{v′}—,
—[C(═O)]ᵥNR₂₁ (CR₂₂R₂₃)ₜ(CR₂₄R₂₅CR₂₈R₂₉O)_{t′}[C(═O)]_{v′}—,
—[C(═O)]ᵥNR₂₁ (CR₂₂R₂₃)ₜ(CR₂₄R₂₅CR₂₈R₂₉O)_{t′}NR₂₆[C(═O)]_{v′}—,

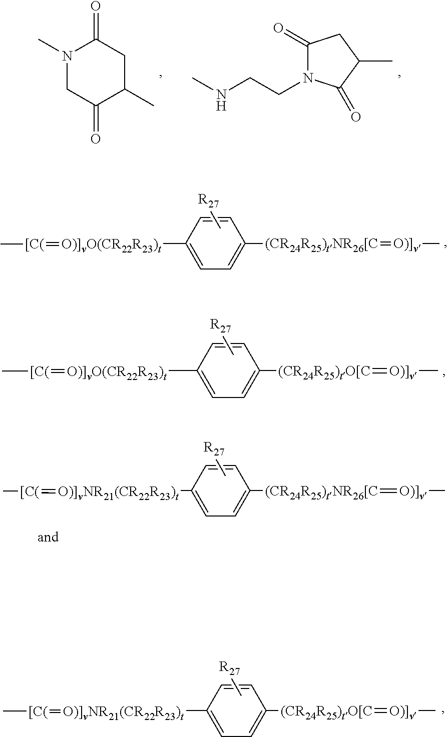

wherein:

R₂₁₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;

(t) and (t′) are independently zero or a positive integer; and

(v) and (v′) are independently zero or 1.

In a further and/or alternative embodiment, bifunctional linkers include an amino acid. The amino acid which can be selected from any of the known naturally-occurring L-amino acids is, e.g., alanine, valine, leucine, isoleucine, glycine, serine, threonine, methionine, cysteine, phenylalanine, tyrosine, tryptophan, aspartic acid, glutamic acid, lysine, arginine, histidine, proline, and/or a combination thereof, to name a few. In alternative aspects, L can be a peptide residue. The peptide can range in size, for instance, from about 2 to about 10 amino acid residues (e.g., 2, 3, 4, 5, or 6).

Derivatives and analogs of the naturally occurring amino acids, as well as various art-known non-naturally occurring amino acids (D or L form), hydrophobic or non-hydrophobic, are also contemplated to be within the scope of the invention. Simply by way of example, amino acid analogs and derivatives include:

2-aminoadipic acid, 3-aminoadipic acid, beta-alanine, beta-aminopropionic acid,

2-aminobutyric acid, 4-aminobutyric acid, piperidinic acid, 6-aminocaproic acid,

2-aminoheptanoic acid, 2-aminoisobutyric acid, 3-aminoisobutyric acid,

2-aminopimelic acid, 2,4-aminobutyric acid, desmosine, 2,2-diaminopimelic acid,

2,3-diaminopropionic acid, N-ethylglycine, N-ethylasparagine, 3-hydroxyproline,

4-hydroxyproline, isodesmosine, allo-isoleucine, N-methylglycine or sarcosine,

N-methylisoleucine, 6-N-methyllysine, N-methylvaline, norvaline, norleucine, ornithine, and others too numerous to mention, that listed in 63 Fed. Reg., 29620, 29622 are incorporated herein by reference.

One embodiment of the L groups includes glycine, alanine, methionine or sarcosine.

Additional linkers are found in Table 1 of Greenwald et al. (Bioorganic & Medicinal Chemistry, 1998, 6:551-562), and in U.S. Pat. Nos. 6,180,095, 6,720,306, 5,965,119, 6,303,569, 6,624,142, 7,122,189, 7,897,647, 7,087,229, and 7,413,738, the contents of each of which are incorporated by reference herein.

### Synthesis of Conjugates of Formula (I)

In the present invention, 1,2-diol groups in the glycan of an C1 inhibitor is oxidized or oxidatively cleaved by employing an oxidizing agent to convert one or two primary OH to an aldehyde or aldehydes. A list of oxidizing agent includes, but is not limited to NaIO₄, H₂O₂, bromine water, chromic acid including pyridinium cidhromate (PDC), pyridinium chlorochromate (PCC), Jones oxidation agent, Collins' reagent, ruthenium oxidizing agent such as tetrapropylammonium perruthenate (TRAP), manganese oxidizing agent such as MnO₂ or KMnO₄, Tollens reagent, or nitric acid. Usual oxidation of an OH group will generate a keto, aldehyde or carboxylic acid. However, some oxidizing agent provides a certain oxidative cleavage for 1,2-diol containing moieties, such as in carbohydrate moiety in glycan of C1 inhibitor. Reacting 1,2-diols with periodic acid would cleave the C—C bond bearing the 1,2-OH moieties and generate two aldehydes as shown below:

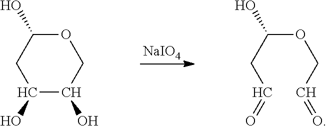

Generally, the conjugates described herein are prepared by reacting the oxidized C1-inhibitor with a polyalkylene oxide having a suitable amine group such as hydrazide or amine, under conditions sufficient to form a covalent bond between the polyalkylene oxide and an aldehyde group in glycan of the C1-esterase inhibitor and purifying the resulting conjugate. Alternatively, the intermediate conjugate is treated further with a reducing agent to form the reduced polymer conjugate, a hydrazide or an alkylated amine.

Polymers are functionalized with nucleophilic functional groups to react with the aldehydes in glycan. The nucleophilic functional group includes, but not limited, amine, or hydrazide. Upon reacting with the aldehyde, the conjugation is achieved by forming a hydrazone bond, which can be optionally reduced to provide a hydrazide bond.

Several examples of conjugation between PEG-hydrazide and the aldehyde in the glycan of C1 inhibitor is provided below.

In one aspect, C1 inhibitor is treated with an oxidizing agent to provide aldehyde, which reacts with amine of polymer such as PEG to form an imine or hydrazone bond first. The imine is reduced by a reducing agent to provide the hydrazide or an amine.

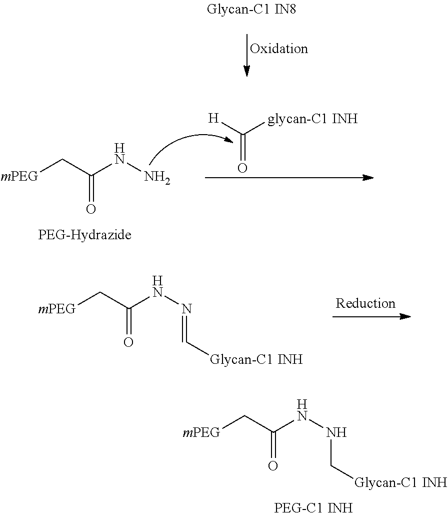

In other embodiment of the present invention, an amine group at the terminal of the polymer reacts with the aldehyde in the presence of a reducing agent to achieve reductive amination to form an amine bond between the polymer and the glycan as shown below.

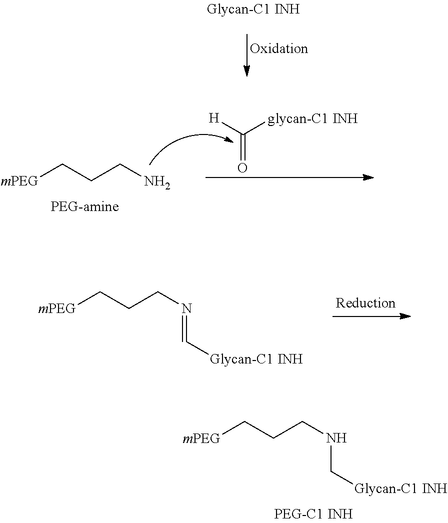

Suitable reducing agents include, for example, sodium cyanoborohydride (NaBH₃CN), sodium triacetoxyborohydride (NaBH(OC(═O)OCH₃)₃), sodium hydride, decaborane (B₁₀H₁₄), InCl₃-Et₃SiH complex, Nickel nanoparticles, Et₃SiH-iridium complex, and Ti(iOPr)₄. One preferable reducing agent is sodium cyanoborohydride.

For purposes of illustration, suitable conjugation reactions include reacting C1-inhibitor with a suitably activated polymer system described herein. The reaction is preferably carried out using conditions well known to those of ordinary skill for protein modification, including the use of a PBS buffered system, etc. with the pH in the range of about 5.0-5.5. It is contemplated that in most instances, an excess of the activated polymer will be reacted with the C1-inhibitor.

Reactions of this sort will often result in the formation of conjugates containing one or more polymers attached to the C1-inhibitor. As will be appreciated, it will often be desirable to isolate the various fractions and to provide a more homogenous product. In most aspects of the invention, the reaction mixture is collected, loaded onto a suitable column resin and the desired fractions are sequentially eluted off with increasing levels of buffer. Fractions are analyzed by suitable analytical tools to determine the purity of the conjugated protein before being processed further.

It will also be appreciated that heterobifunctional polyalkylene oxides are also contemplated for purposes of cross-linking C1-inhibitor, or providing a means for attaching other moieties such as targeting agents for conveniently detecting or localizing the polymer-C1-inhibitor conjugate in a particular areas for assays, research or diagnostic purposes.

### Formulations

Polymer conjugates of the present invention may be manufactured and formulated by processes well known in the art, e.g., using a variety of well-known mixing, dissolving, granulating, levigating, emulsifying, encapsulating, entrapping or lyophilizing processes. Compositions may be formulated in conjunction with one or more physiologically acceptable carriers comprising excipients and auxiliaries which facilitate processing of the active conjugates into preparations which can be used pharmaceutically. Proper formulation is dependent upon the route of administration chosen. Parenteral routes are preferred in many aspects of the invention, but not limited to.

In another aspect, the conjugates may also be formulated for parenteral administration or injection, e.g., by bolus injection or continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. For injection, including, without limitation, intravenous, intramuscular and subcutaneous injection, the polymer conjugates of the invention may be formulated in aqueous solutions, preferably in physiologically compatible buffers such as physiological saline buffer or polar solvents including, without limitation, a pyrrolidone or dimethylsulfoxide. Aqueous injection suspensions may contain substances that increase the viscosity of the suspension, such as sodium carboxymethyl cellulose, sorbitol, or dextran. Additionally, suspensions of the active conjugates may be prepared in a lipophilic vehicle. Suitable lipophilic vehicles include fatty oils such as sesame oil, synthetic fatty acid esters such as ethyl oleate and triglycerides, or materials such as liposomes. Optionally, the suspension may also contain suitable stabilizers and/or agents that increase the solubility of the conjugates to allow for the preparation of highly concentrated solutions. Alternatively, the active ingredient may be in powder form, such as lyophilized product, for constitution with a suitable vehicle, e.g., sterile, pyrogen-free water, before use.

### Methods of Administration and Dosage

The C1-inhibitor polymer conjugate described herein is useful for all of the methods and indications already art-known for Cinryze® (Viro Pharma Biologics, Inc.) and Berinert® (CSL Behring LLC). Thus, the inventive C1-inhibitor conjugate is administered to a patient in need thereof in an amount that is effective to treat a disease or disorder or other condition that is responsive to such treatment. The artisan will appreciate suitable amounts, routes of administration and dosing schedules extrapolated from the known properties of Cinryze® and Berinert®.

Another aspect of the present invention provides methods of treatment for various medical conditions in mammals, preferably humans. The methods include administering an effective amount of a pharmaceutical composition that includes a C1-inhibitor polymer conjugate prepared as described herein, to a mammal in need of such treatment. The conjugates are useful for, among other things, treating C1-inhibitor-susceptible conditions or conditions which would respond positively or favorably as these terms are known in the medical arts to C1-inhibitor-based therapy.

Conditions that can be treated in accordance with the present invention are generally those that are susceptible to treatment with C1-inhibitor. Exemplary conditions which can be treated with C1-inhibitor include, but are not limited to, ongoing, acute attacks of hereditary angioedema (HAE) affecting the abdomen, face or throat in adults and adolescents and all other medical conditions know to those of ordinary skill to benefit from C1-inhibitor therapy. In a preferred aspect of the invention, the polymer conjugated C1-inhibitor is administered to patients in amounts effective to treat hereditary angioedema or prevent swelling and/or painful attacks in teenagers and adults with Hereditary Angioedema.

Administration of the described dosages may be every other day, but is preferably once or twice a week. Doses are usually administered over at least a 24 week period by injection or infusion. Administration of the dose can be intravenous, subcutaneous, intramuscular, or any other acceptable systemic method, including subdermal or transdermal injection via conventional medical syringe and/or via a pressure system. Based on the judgment of the attending clinician, the amount of drug administered and the treatment regimen used will, of course, be dependent on the age, sex and medical history of the patient being treated, the stage or severity of the specific disease condition and the tolerance of the patient to the treatment as evidenced by local toxicity and by systemic side-effects. Dosage amount and frequency may be determined during initial screenings of neutrophil count.

The amount of the C1-inhibitor polymer conjugate composition administered to treat the conditions described above is based on the C1-inhibitor activity of the polymeric conjugate. It is an amount that is sufficient to significantly affect a positive clinical response. Although the clinical dose will cause some level of side effects in some patients, the maximal dose for mammals including humans is the highest dose that does not cause unmanageable clinically-important side effects. For purposes of the present invention, such clinically important side effects are those which would require cessation of therapy due to severe flu-like symptoms, central nervous system depression, severe gastrointestinal disorders, alopecia, severe pruritus or rash. Substantial white and/or red blood cell and/or liver enzyme abnormalities or anemia-like conditions are also dose limiting.

A therapeutically effective amount refers to an amount of conjugate effective to prevent, alleviate or ameliorate the C1-inhibitor-susceptible condition. Determination of a therapeutically effective amount is well within the capability of those skilled in the art, especially in light of the disclosure herein.

The dosage, of course, can vary depending upon the dosage form and route of administration. The exact formulation, route of administration and dosage can be selected by the individual physician in view of the patient's condition.

For any conjugate used in the methods of the invention, the therapeutically effective amount may be estimated initially from in vitro assays. Then, the dosage can be formulated for use in animal models so as to achieve a circulating concentration range that includes the effective dosage. Such information can then be used to more accurately determine dosages useful in patients.

Toxicity and therapeutic efficacy of the conjugates described herein can be determined by standard pharmaceutical procedures in cell cultures or experimental animals using methods well-known in the art.

As explained above, the dosages of the polymer C1-inhibitor conjugate compositions of the present invention will vary somewhat depending upon the C1-inhibitor moiety and polymer selected. In general, however, the conjugate is administered in amounts ranging from about 100 to about 5,000 u/kg/week, from about 500 to about 4,000 u/kg/week or from about 1,000 to 3,000 u/kg/week of C1-inhibitor equivalent in the polymer conjugate, based on the condition of the treated mammal or human patient. The range set forth above is illustrative and those skilled in the art will determine the dosing of the conjugate selected based on clinical experience and the treatment indication.

The conjugates may be administered once daily or divided into multiple doses which can be given as part of a multi-week treatment protocol. The precise dose will depend on the stage and severity of the condition, the susceptibility of the condition to the C1-inhibitor polymer conjugate, and the individual characteristics of the patient being treated, as will be appreciated by one of ordinary skill in the art.

Practice of the invention would allow treatment of this condition, and others, at higher doses and in combination with other art-known therapeutic agents.

### EXAMPLES

The following examples serve to provide further appreciation of the invention but are not meant in any way to restrict the effective scope of the invention.

### Materials

Reagents: C1 Esterase Inhibitor was obtained from Athens Research & Technology and have MW: 73000 Da as determined by MALDI. Activated PEG's were obtained from NOF;
Buffers: (1) 100 mM Na acetate, 150 mM naCl, pH5.5; (2) PBS

### Ultrafiltration: 10 k Pellicon XL 50 Ultrafiltration Cassettes

### Amicon Membrane: 30K Ultrafiltration Membrane (Millipore)

Sterile Filter: 0.2 μm sterile polyethersulfone filter (VWR)

### Purification of Mono and Di PEGylated C1 INH Conjugates

Mono or Di PEGylated C1-INH (both PEG linear and branched) was purified by weak anion exchange column (HiTrap DEAE FF, 1 ml. GE Healthcare) or by hydrophobic interaction column (HIC phenyl FF, 1 ml. GE Healthcare). In DEAE column purification, Buffer A contained 10 mM Tris, pH 8.5 and buffer B had 0.5 M NaCl in buffer A. Elution was conducted at 1 ml/min over 30 min. Based on SDS-PAGE, the majority components in flow through was di PEG-C1 INH. Mono PEG-C1 INH and native C1 INH were both bound to the column and started to elute out at 0.12 M NaCl. The fractions containing mono PEG-C1 INH identified by SDS-PAGE was concentrated using Centricon YM30 (Millipore) and the buffer was exchanged to PBS by NAP-5 column (GE Healthcare). In HIC phenyl purification, Buffer A contained 0.75 M ammonium sulfate in PBS buffer and buffer was PBS. Elution was conducted at 1 ml/min over 30 min. The first elution peak identified on SDS-PAGE was mono PEG-C1 INH and second peak was di PEG-C1 INH. Mono and di PEG-C1 INH were concentrated using Centricon YM30 and buffer-exchanged to PBS by NAP-5 column.

### Example 1

### C1 INH PEGylation at Glycan-Hydrazide

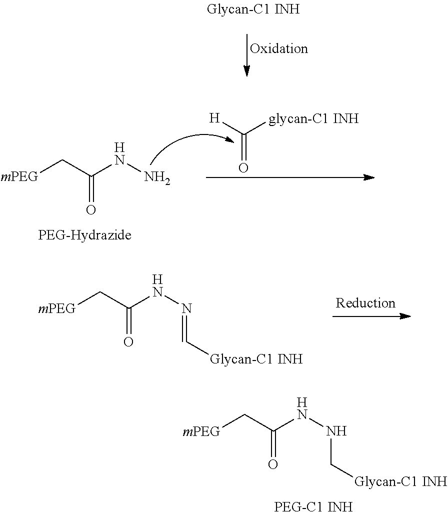

C1 INH (60 mg) was suspended in 100 mM sodium phosphate, pH 6.0, 150 mM sodium chloride at 2 mg/ml concentration. Sodium periodate was added to a final concentration of 10 mM. The oxidation reaction was conducted at 23° C. for 15 min. The excess of sodium periodate was removed on G-25 desalting column. PEG-hydrazide (10, 20, or 30k) was added to a reaction molar ratio of higher than 30:1. The reaction was conducted in the presence or absence of sodium cyanoborohydride at 4° C. for 18 hours. Unreacted aldehyde could be quenched with glycine, Tris buffer, or ethanolamine. Excess of PEG was removed by size exclusion chromatography such as Sephacryl S-400 column equilibrated in PBS. The overall yield of purification was 67% (60 mg was down to 52 mg after desalting to remove sodium periodate and further down to 40 mg after SEC Sephacryl S400). The purified conjugates contained 11% free PEG as analyzed by SEC-MALS.

### Example 2

### Characterization of PEG-C1 INH

The concentration of PEGylated C1 INH was determined by UV at 280 nm (extinction coefficient of C1 INH was 0.39 mL/mg·cm). Sample at 5 μg or 10 μg was loaded into the gel without sample reduction and heating for electrophoresis. In a separate experiment, the samples were heated at 70° C. for 7 minutes in the presence or absence of 75 mM β-mercaptoethanol in gel buffer. There was no evidence of conjugate breakdown under such conditions. However, when the sample was treated with acid such as 0.1% TFA for reverse phase HPLC or sodium acetate, pH 4.7 for purification, PEG was observed to fall off. The protein bands were visualized after simple blue stain. The density of the image was obtained on Molecular Dynamics. As seen on SDS gel, all C1 INH was converted into PEGylated form. There were eight PEG (20 or 30k) strands attached per C1 INH as analyzed SEC-MALS.

### Example 3

### C1 INH Activity Assay

C1 INH activity was measured by the inhibition of C1 esterase and kallikrein activities. For the inhibition of C1 esterase activity, samples, standards, and controls were added to 96-well plate, and then C1-esterase was added. After 10 min incubation at 37° C., substrate of C1-eserasse was added. C1-esterase activity for cleavage of the substrate was monitored at 37° C. for 4 minutes kinetically. The higher C1 INH activity results in the lower C1 esterase activity or the lower cleavage rate of substrate. For the inhibition of kallikrein activity, EC₅₀ was used to evaluate PEG-C1 INH activity. Lower EC₅₀ value indicates higher activity of kallikrein inhibition.

| | | Activity of | | Activity of | |
|---|---|---|---|---|---|
| | | C1 Esterase | | Kallikrein | |
| | | inhibition | | inhibition | |
| | PEG#/ | | % C1 INH | EC₅₀ | % C1 INH |
| Compound | C1 INH | U/mg | activity | (μM) | activity |
| C1 INH | — | 7.0 | 100 | 0.2 | 100 |
| PEG-20k-Glycan-C1 | 8 | 5.1 | 73 | 0.3 | 67 |
| INH (60 mg scale) | | | | | |
| PEG-20k-Glycan-C1 | pending | 6.6 | 94 | | |
| INH (5 mg scale) | | | | | |

PEGylation of C1 INH at glycan was achieved. All C1 INH had been converted into the PEGylated form and there were eight PEG-20k strands attached per C1 INH. The PEG-20k-C1 INH conjugate retained 73% C1 INH activity on C1 esterase inhibition (97% in small scale) and 67% C1 INH activity on Kallikrein inhibition (the highest activity among seven PEG-C1 INH candidates). The conjugation yield was 100% and the purification yield was 67%, although 11% free PEG was detected by SEC-MALS. The conjugate was stable at neutral pH to heating and reducing as visualized on SDS gel and became releasable under acidic conditions. Cleavage of hydrazone bond formed between PEG-hydrazide and protein-aldehyde under acidic conditions may represent a new type of releasable linker.

### Example 4

### In Vivo Pharmacokinetics

The polymeric conjugates of C1 inhibitor prepared is administered (i.v.) to groups of rat for in vivo plasma pharmacokinetic (PK) study at dose of 70 U/kg. The polymer conjugates of the invention demonstrate improved half-lives compared to the native C1-esterase inhibitor. Some polymer conjugates have an extended half-life to about 80 hours, a more than 10 fold improvement than the native C1 inhibitor. This profile suggests a long lasting treatment regime such as once a week.

1. A polymer conjugate, comprising:
a C1-inhibitor having at least one substantially non-antigenic polymer covalently attached thereto via glycan moiety of the C1-inhibitor.

2. The polymer conjugate of claim 1, wherein the substantially non-antigenic polymer is a polyalkylene oxide.

3. The polymer conjugate of claim 2, wherein the polyalkylene oxide is PEG

4. The polymer conjugate of claim 1, wherein the C1-inhibitor is a human C1 esterase inhibitor (C1-INH).

5. The polymer conjugate of claim 1, wherein the C1-inhibitor is a polypeptide represented by SEQ ID NO:1 or SEQ ID NO:2.

6. The polymer conjugate of claim 1, wherein one of the substantially non-antigenic polymer is attached to an aldehyde in the glycan, wherein the aldehyde is generated by oxidation.

7. (canceled)

8. The polymer conjugate of claim 1, wherein one of the substantially non-antigenic polymer contains a hydrazide or an amine.

9. (canceled)

10. The polymer conjugate of claim 1, wherein the polymer conjugate retains about 40-80% of the biological activity of the C1-inhibitor in its native form.

11. (canceled)

12. The polymer conjugate of claim 1, wherein the molecular weight of the substantially non-antigenic polymer ranges from about 2,000 to about 100,000 daltons.

13. The polymer conjugate of claim 1, wherein the substantially non-antigenic polymer is conjugated via hydrazone, hydrazide, imine, or an amine.

14. The polymer conjugate of claim 3, wherein the conjugate comprises Formula (I) or (I′):
[PEG-(L)ₘ-(C(═Y)—NH)ₙ]ₚ—(X)_{p′}—C1-inhibitor (I) or
[PEG-(L)ₘ-(C(═Y)—NH)ₙ]ₚ—(X)_{p′}—C1-inhibitor-(X)_{q′}-[(C(═Y′)—NH)_{n′}-(L′)_{m′}-PEG]_{q′} (I′)
wherein
C1 inhibitor is bonded to PEG via an amine from PEG through glycan site;
PEG is a linear, branched or multi-arm poly(ethylene glycol) having a terminal group —(CH₂CH₂O)—;
Y or Y′ is independently O or S;
L or L′ is independently a linker or functional group suitable to react with thiol;
(m) or (m′) is independently 0 or 1;
(n) or (n′) is independently zero or a positive integer, preferably selected from 1, 2, 3, 4, 5, 6, 7, 8, 9, or 10;
(p) or (q) is independently a positive integer, preferably selected from 1, 2, 3, 4, 5, 6 or 7; and
X or X′ is NH, an amine from PEG hydrazine or PEG amine;
(p′) or (q′) is independently a positive integer same as (p) or (q), respectively, provided that (m), (m′), (n) and (n′) are not zero simultaneously.
In one aspect of the invention, in the polymer conjugate of Formula (I) or (I′) described above, (n) or (n′) is a positive integer selected from among 1, 2, 3, 4, 5, 6 or 7 and (p) or (q) is a positive integer selected from among of 1, 2 or 3.

15. (canceled)

16. The polymer conjugate of claim 14, wherein L is selected from the group consisting of:
a natural L-amino acid or its derivatives;
—[C(═O)]ᵥ(CR₂₂R₂₃)ₜ[C(═O)]_{v′}—,
—[C(═O)]ᵥ(CR₂₂R₂₃)ₜ—O[C(═O)]_{v′}—,
—[C(═O)]ᵥ(CR₂₂R₂₃)ₜ—NR₂₆[C(═O)]_{v′}—,
—[C(═O)]ᵥO(CR₂₂R₂₃)ₜ[C(═O)]_{v′}—,
—[C(═O)]ᵥO(CR₂₂R₂₃)ₜO[C(═O)]_{v′}—,
—[C(═O)]ᵥO(CR₂₂R₂₃)ₜNR₂₆[C(═O)]_{v′}—,
—[C(═O)]ᵥNR₂₁ (CR₂₂R₂₃)ₜ[C(═O)]_{v′}—,
—[C(═O)]ᵥNR₂₁ (CR₂₂R₂₃)ₜO[C(═O)]_{v′}—,
—[C(═O)]ᵥNR₂₁ (CR₂₂R₂₃)ₜNR₂₆[C(═O)]_{v′}—,
—[C(═O)]ᵥ(CR₂₂R₂₃)ₜO(CR₂₈R₂₉)_{t′}[C(═O)]_{v′}—,
—[C(═O)]ᵥ(CR₂₂R₂₃)ₜNR₂₆—(CR₂₈R₂₉)_{t′}[C(═O)]_{v′}—,
—[C(═O)]ᵥ(CR₂₂R₂₃)ₜS—(CR₂₈R₂₉)_{t′}[C(═O)]_{v′}—,
—[C(═O)]ᵥO(CR₂₂R₂₃)ₜO(CR₂₈R₂₉)_{t′}[C(═O)]_{v′}—,
—[C(═O)]ᵥO(CR₂₂R₂₃)ₜNR₂₆—(CR₂₈R₂₉)_{t′}[C(═O)]_{v′}—,
—[C(═O)]ᵥO(CR₂₂R₂₃)ₜS—(CR₂₈R₂₉)_{t′}[C(═O)]_{v′}—,
—[C(═O)]ᵥNR₂₁ (CR₂₂R₂₃)ₜO—(CR₂₈R₂₉)_{t′}[C(═O)]_{v′}—,
—[C(═O)]ᵥNR₂₁ (CR₂₂R₂₃)ₜNR₂₆—(CR₂₈R₂₉)_{t′}[C(═O)]_{v′}—,
—[C(═O)]ᵥNR₂₁ (CR₂₂R₂₃)ₜS—(CR₂₈R₂₉)_{t′}[C(═O)]_{v′}—,
—[C(═O)]ᵥ(CR₂₂R₂₃CR₂₈R₂₉O)ₜNR₂₆[C(═O)]_{v′}—,
—[C(═O)]ᵥ(CR₂₂R₂₃CR₂₈R₂₉O)ₜ[C(═O)]_{v′}—,
—[C(═O)]ᵥO(CR₂₂R₂₃CR₂₈R₂₉O)ₜNR₂₆[C(═O)]_{v′}—,
—[C(═O)]ᵥO(CR₂₂R₂₃CR₂₈R₂₉O)ₜ[C(═O)]_{v′}—,
—[C(═O)]ᵥNR₂₁ (CR₂₂R₂₃CR₂₈R₂₉O)ₜNR₂₆[C(═O)]_{v′}—,
—[C(═O)]ᵥNR₂₁ (CR₂₂R₂₃CR₂₈R₂₉O)ₜ[C(═O)]_{v′}—,
—[C(═O)]ᵥ(CR₂₂R₂₃CR₂₈R₂₉O)ₜ(CR₂₄R₂₅)_{t′}[C(═O)]_{v′}—,
—[C(═O)]ᵥO(CR₂₂R₂₃CR₂₈R₂₉O)ₜ(CR₂₄R₂₅)_{t′}[C(═O)]_{v′}—,
—[C(═O)]ᵥNR₂₁ (CR₂₂R₂₃CR₂₈R₂₉O)ₜ(CR₂₄R₂₅)_{t′}[C(═O)]_{v′}—,
—[C(═O)]ᵥ(CR₂₂R₂₃CR₂₈R₂₉O)ₜCR₂₄R₂₅)_{t′}O[C(═O)]_{v′}—,
—[C(═O)]ᵥ(CR₂₂R₂₃)ₜ(CR₂₄R₂₅CR₂₈R₂₉O)_{t′}[C(═O)]_{v′}—,
—[C(═O)]ᵥ(CR₂₂R₂₃)ₜ(CR₂₄R₂₅CR₂₈R₂₉O)_{t′}NR₂₆[C(═O)]_{v′}—,
—[C(═O)]ᵥO(CR₂₂R₂₃CR₂₈R₂₉O)ₜ(CR₂₄R₂₅)_{t′}O[C(═O)]_{v′}—,
—[C(═O)]ᵥO(CR₂₂R₂₃)ₜ(CR₂₄R₂₅CR₂₈R₂₉O)_{t′}[C(═O)]_{v′}—,
—[C(═O)]ᵥO(CR₂₂R₂₃)ₜ(CR₂₄CR₂₅CR₂₈R₂₉O)_{t′}NR₂₆[C(═O)]_{v′}—,
—[C(═O)]ᵥNR₂₁ (CR₂₂R₂₃CR₂₈R₂₉O)ₜ(CR₂₄R₂₅)_{t′}O[C(═O)]_{v′}—,
—[C(═O)]ᵥNR₂₁ (CR₂₂R₂₃)ₜ(CR₂₄R₂₅CR₂₈R₂₉O)_{t′}[C(═O)]_{v′}—,
—[C(═O)]ᵥNR₂₁ (CR₂₂R₂₃)ₜ(CR₂₄R₂₅CR₂₈R₂₉O)_{t′}NR₂₆[C(═O)]_{v′}—, 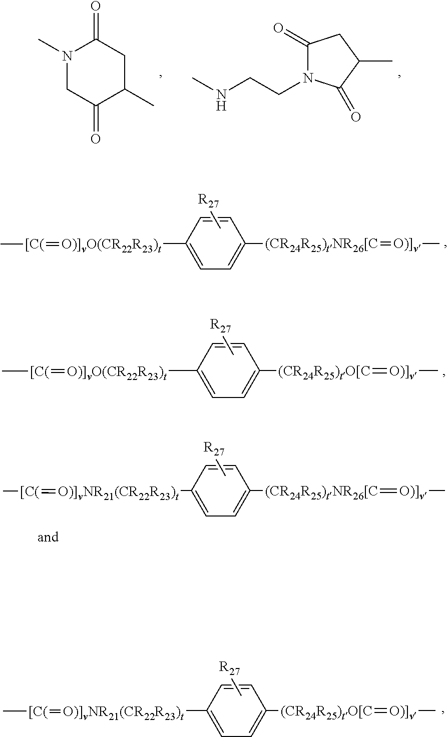
wherein:
R₂₁₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
(t) and (t′) are independently zero or a positive integer; and
(v) and (v′) are independently zero or 1.

17. The polymer conjugate of claim 3 selected from the group consisting of: 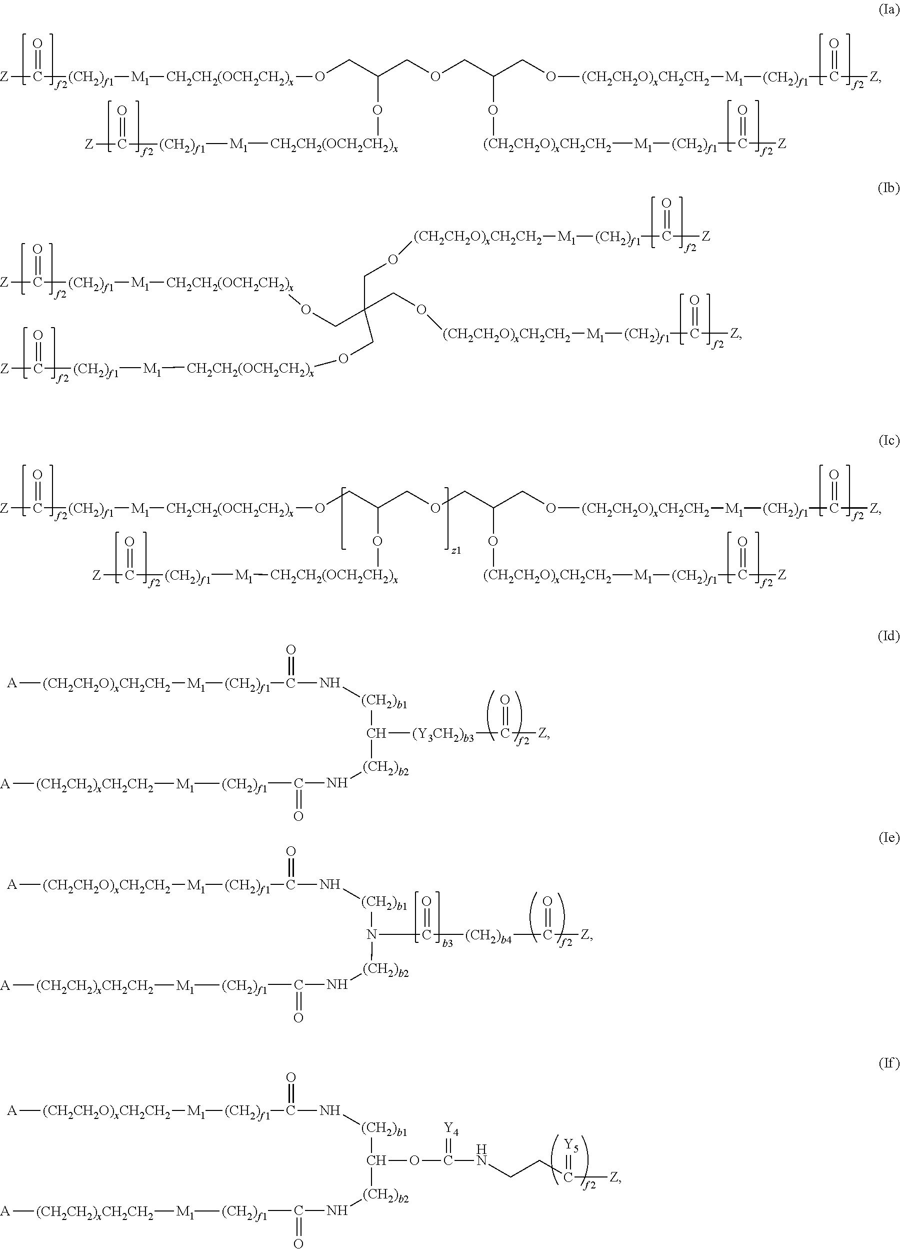 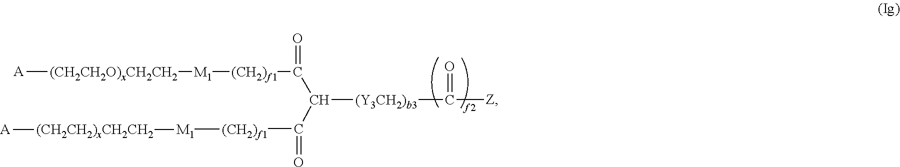
Z—[C(═O)]_{f2}—(CH₂)_{f1}-M₁-CH₂CH₂—O—(CH₂CH₂O)ₓ—CH₂CH₂-M₁-(CH₂)_{f1}-[C(═O)]_{f2}—Z, and (Ih)
A-(CH₂CH₂O)ₓ—CH₂CH₂-M₁-(CH₂)_{f1}-[C(═O)]_{f2}—Z, (Ii)
wherein
A is hydroxyl, NH₂, CO₂H, or C₁₋₆ alkoxy;
M₁ is O, S, or NH;
Y₃ is O, NR₅₁, S, SO or SO₂;
Y₄ and Y₅ are independently O, S or NR₅₁;
R₅₁, in each occurrence, is independently hydrogen, C₁₋₈ alkyl, C₁₋₈ branched alkyl, C₁₋₈ substituted alkyl, aryl, or aralkyl;
Z, in each occurrence, is independently OH, a leaving group, a targeting group, C₁₋₈ alkyl, C₁₋₈ alkoxy or C1 inhibitor containing moiety;
(b1) and (b2) are independently zero or positive integers;
(b3) is zero or 1;
(b4) is a positive integer;
(f1) is zero or a positive integer of from about 1 to about 10;
(f2) is zero or 1;
(z1) is zero or a positive integer of from 1 to about 27;
(x) is a degree of polymerization positive integer of from about 10 to about 2,300 so that the polymeric portion of the compound has the total number average molecular weight of from about 2,000 to about 100,000 daltons, provided that one or more Z are C1 inhibitor containing moiety.

18. The polymer conjugate of claim 3 selected from the group consisting of: 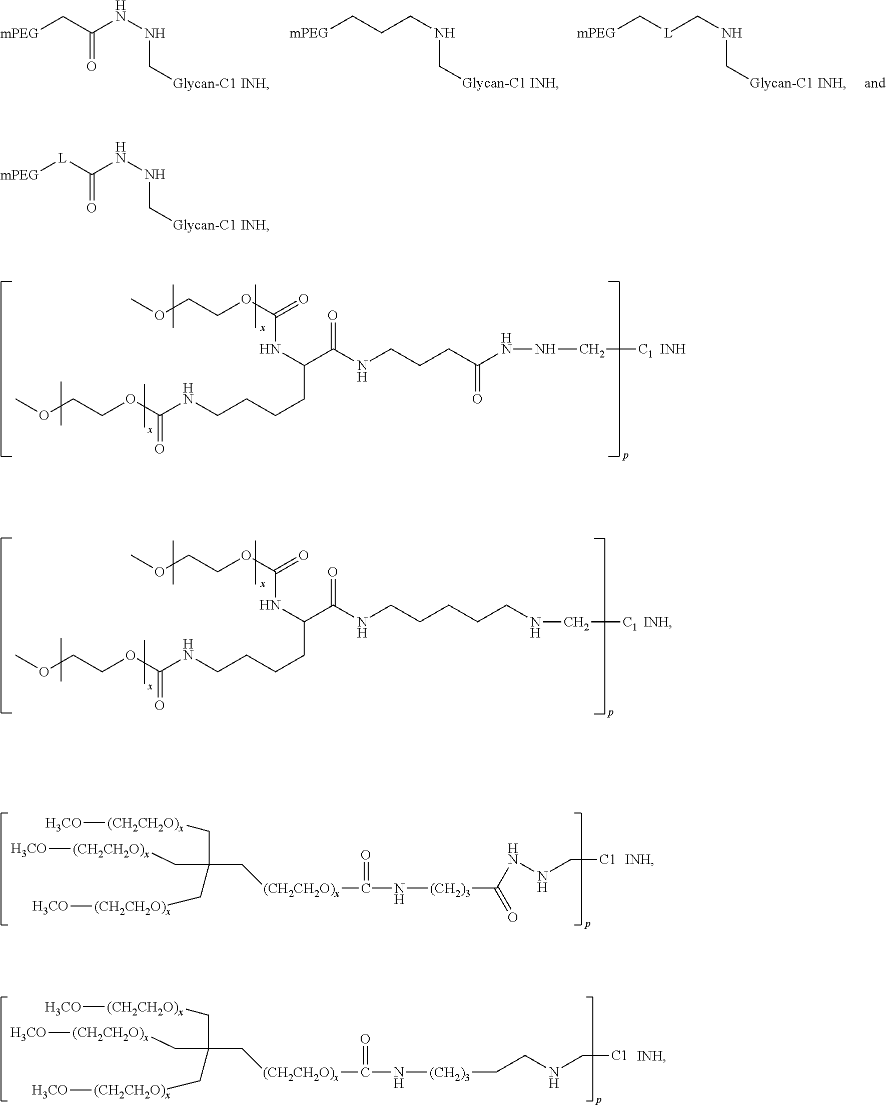 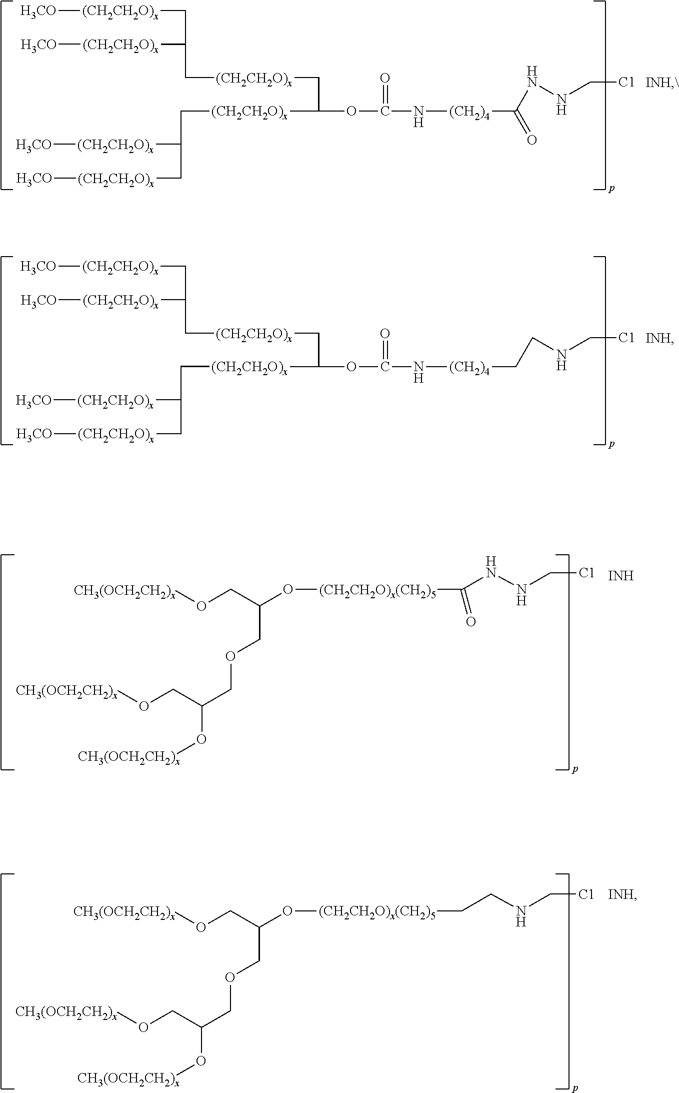 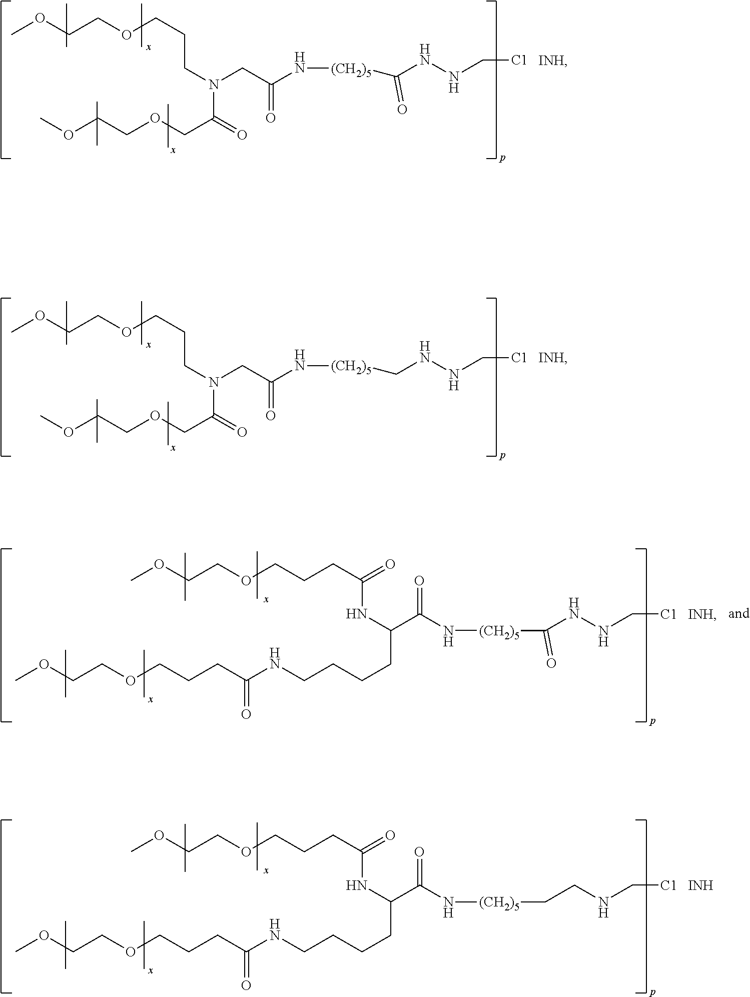
wherein,
C1 INH is C1 inhibitor bonded to the polymer through a glycan moiety in C1 inhibitor;
mPEG is CH₃O—(CH₂CH₂O)ₓ—;
(x) is a degree of polymerization positive integer of from about 10 to about 2,300 so that the polymeric portion of the compound has the total number average molecular weight of from about 2,000 to about 100,000 daltons; and
(p) is a positive integer.

19. A method of preparing a polymer conjugate comprising a C1-esterase inhibitor having at least one polyalkylene oxide thereto via glycan moiety of the C1-inhibitor, the method comprising:
reacting C1-esterase inhibitor with a polyalkylene oxide having an activating group, under conditions sufficient to form a covalent bond between the polyalkylene oxide and an aldehyde group in glycan of the C1-esterase inhibitor; and
purifying the resulting conjugate.

20. The method of claim 19, wherein the activating group is an amine or a hydrazide and the reaction is carried out in the presence of a reducing agent.

21. The method of claim 19, wherein the activating group is a hydrazide

22. A method of treating a mammal comprising administering an effective amount of a polymer conjugate of claim 1 to a patient in need thereof.

23. The method of claim 21, wherein the polymer conjugate is administered in amounts from about 100 u/kg/week to about 5,000 u/kg/week of C1-inhibitor equivalent in the polymer conjugate.

24. The method of claim 21, wherein the polymer conjugate is administered in amounts from about 500 u/kg/week to about 4000 u/kg/week of C1-inhibitor equivalent in the polymer conjugate.
